(19) United States
(12) Patent Application Publication
(10) Pub. No.: US 2005/0118625 A1
(43) Pub. Date: Jun. 2, 2005

(54) Nucleic acid arrays for detecting gene expression associated with human osteoarthritis and human proteases

(76) Inventor: William Martin Mounts, Andover, MA (US)

Correspondence Address: NIXON PEABODY, LLP, 401 9TH STREET, NW, SUITE 900, WASHINGTON, DC, 20004-2128, US

(21) Appl. No.: 10/956,157

(22) Filed: Oct. 4, 2004

Related U.S. Application Data
(60) Provisional application No. 60/507,511, filed on Oct. 2, 2003.

(51) Int. Cl. C12Q 1/68; C12M 1/34

(52) U.S. Cl. 435/6; 435/287.2

(57) ABSTRACT

The present invention provides nucleic acid arrays and methods of using the same for expression profiling of human protease and/or osteoarthritis genes. The nucleic acid arrays of the present invention include one or more substrate supports. A substantial portion of all polynucleotide probes that are stably attached to the substrate support(s) can hybridize under stringent or nucleic acid array hybridization conditions to human protease or osteoarthritis genes. In one embodiment, the nucleic acid arrays of the present invention include a plurality of probe sets, each of which can hybridize under stringent or nucleic acid array hybridization conditions to a different respective tiling sequence selected from Attachment C, or the complement thereof.

### RELATED APPLICATIONS

This application claims benefit and incorporates by reference the entire disclosure of U.S. Provisional Application Ser. No. 60/507,511 filed Oct. 2, 2003.

All materials on the compact discs labeled “Copy 1” and “Copy 2” are incorporated herein by reference in their entireties. Each of the compact discs includes the following files: Attachment A.txt (48 KB, created Mar. 9, 2004), Attachment B.txt (728 KB, created Mar. 9, 2004), Attachment C.txt (767 KB, created Mar. 10, 2004), Attachment D.txt (97 KB, created Mar. 10, 2004), Attachment E.txt (7,768 KB, created Oct. 3, 2004), Attachment F.txt (1,039 KB, created Mar. 15, 2004), Attachment G.txt (20 KB, created Mar. 15, 2004), Attachment H.txt (11,402 KB, created Oct. 3, 2004), and Sequence Listing.ST25.txt (65,689 KB, created Sep. 27, 2004).

### TECHNICAL FIELD

This invention relates to nucleic acid arrays and methods of using the same for detecting gene expression associated with human osteoarthritis and human proteases.

### BACKGROUND

Osteoarthritis is one of the most common diseases of the elderly. It mostly affects the weight-bearing joints such as spine, knees and hips, but thumb and finger joints may also be affected. Osteoarthritis is mainly a disease of “wear and tear.” Repetitive mechanical injury of the cartilage eventually results in loss of cartilage and damage to joint surfaces and adjacent bone. Inflammatory cells then invade the damaged joints, causing pain, swelling and stiffness of the joints. The repetitive mechanical injury also leads to pathological changes that are characterized by the loss of proteoglycans and collagen from the cartilage matrix.

Cartilage cells, such as chondrocytes, produce proteoglycans and collagen to form the cartilage matrix at joints. Cartilage cells also secrete a number of proteases to degrade the matrix structure. These proteases include collagenase, gelatinase, stromelysin, cathepsin, tissue plasminogen activator, and other metalloproteinases, cysteine proteases, aspartic proteases and serine proteases. The activities of these proteases are regulated by protease inhibitors, such as plasminogen activator inhibitors and numerous tissue inhibitors of metalloproteinase. The imbalance between the levels of the proteases and their inhibitors is believed to contribute to the onset and progress of osteoarthritis.

### SUMMARY OF THE INVENTION

The present invention provides nucleic acid arrays and methods of using the same for detecting gene expression associated with human osteoarthritis and human proteases. The nucleic acid arrays of the present invention are concentrated with probes for human protease genes and/or genes that are differentially expressed in osteoarthritic human cartilage cells as compared to osteoarthritis-free human cartilage cells. By concentrating probes for these genes on a single array, the present invention facilitates the study on human osteoarthritis and accelerates the drug development process for the treatment of osteoarthritis and other protease-related diseases.

In one aspect, a nucleic acid array of the present invention comprises one or more substrate supports. A substantial portion of all polynucleotide probes that are stably associated with the substrate support(s) can hybridize under stringent or nucleic acid array hybridization conditions to human protease genes and/or genes that are differentially expressed in osteoarthritic human cartilage cells as compared to osteoarthritis-free human cartilage cells. The differentially expressed genes can include genes whose expression is substantially elevated in osteoarthritic cartilage cells relative to osteoarthritis-free cartilage cells. The differentially expressed genes can also include genes whose expression is substantially reduced in osteoarthritic cartilage cells relative to osteoarthritis-free cartilage cells.

In one embodiment, a substantial portion of all polynucleotide probes on a nucleic acid array of the present invention comprises one or more first probe sets, and each of these first probe sets is capable of hybridizing under stringent or nucleic acid array hybridization conditions to a gene whose average expression level in osteoarthritic human cartilage cells is higher or substantially higher than that in osteoarthritis-free human cartilage cells. In another embodiment, the substantial portion of all polynucleotide probes further comprises one or more second probe sets, and each of these second probe sets is capable of hybridizing under stringent or nucleic acid array hybridization conditions to a gene whose average expression level in osteoarthritis-free human cartilage cells is higher or substantially higher than that in osteoarthritic human cartilage cells. In still another embodiment, the substantial portion of all polynucleotide probes further comprises one or more third probe sets, and each of these third probe sets is capable of hybridizing under stringent or nucleic acid array hybridization conditions to a human protease gene. As used herein, a probe set can hybridize to a target gene if each probe in the probe set can hybridize to the target gene (e.g., an mRNA, cDNA or codon sequence of the gene, or the complement thereof).

In one example, a substantial portion of all polynucleotide probes on a nucleic acid array of the present invention comprises (a) at least 2, 5, 10, 100, 500, or more first probe sets, each of which is capable of hybridizing under stringent or nucleic acid array hybridization conditions to a different respective gene whose average expression level in osteoarthritic human cartilage cells is substantially higher than that in osteoarthritis-free human cartilage cells; (b) at least 2, 5, 10, 100, 500, or more second probe sets, each of which is capable of hybridizing under stringent or nucleic acid array hybridization conditions to a different respective gene whose average expression level in osteoarthritis-free human cartilage cells is substantially higher than that in osteoarthritic human cartilage cells; and (c) at least 2, 5, 10, 100, 500, or more third probe sets, each of which is capable of hybridizing under stringent or nucleic acid array hybridization conditions to a different respective human protease gene. By “different respective”, it means that each probe set in a group of probe sets can hybridize to a gene that is different from those to which other probe sets in the group hybridize. Each probe set can include any number of probes, such as 2, 5, 10, 15, 20, 25, or more.

In yet another embodiment, a substantial portion of all polynucleotide probes on a nucleic acid array of the present invention includes at least 15%, 25%, 35%, 45%, or more of all polynucleotide probes that are stably associated with the substrate support(s) of the nucleic acid array.

In another embodiment, a nucleic acid array of the present invention includes at least 1, 2, 3, 4, 5, 10, 50, 100, 500, 1,000, 5,000, or more probe sets, each of which can hybridize under stringent or nucleic acid array hybridization conditions to a different respective tiling sequence selected from Attachment C, or the complement thereof. In one example, these probe sets constitutes a substantial portion of all polynucleotide probes that are stably associated with the nucleic acid array. In another example, the nucleic acid array includes at least 5,028 probe sets, each of which can hybridize under stringent or nucleic acid array hybridization conditions to a different respective tiling sequence selected from Attachment C, or the complement thereof. In still another example, the nucleic acid array includes each and every oligonucleotide probe selected from Attachment E. In many cases, the nucleic acid arrays of the present invention include a perfect mismatch probe for each perfect match probe.

The present invention also features methods of screening for candidate drugs capable of modulating expression of human protease or osteoarthritis genes. In one embodiment, the methods comprise the steps of:
(a) preparing a first nucleic acid sample from a human affected by osteoarthritis;
(b) hybridizing the first nucleic acid sample to a first nucleic acid array of the present invention;
(c) detecting a first set of hybridization signals;
(d) treating the human with a candidate drug;
(e) repeating steps (a)-(c) with a second nucleic acid sample from the treated human and a second nucleic acid array identical to the first array to obtain a second set of hybridization signals; and
(f) comparing the first and second sets of hybridization signals, where any change in expression level of at least one protease gene, and/or one gene differentially expressed in osteoarthritic human cartilage cells as compared to osteoarthritis-free human cartilage cells, identifies the candidate drug as one that modulates expression of human protease or osteoarthritis genes. In many cases, the first and second nucleic acid samples are prepared from cartilage tissues.

In another embodiment, the methods of the present invention comprise the steps of:
(a) preparing a first nucleic acid sample from a cell or tissue affected by osteoarthritis;
(b) hybridizing the first nucleic acid sample to a first nucleic acid array of the present invention;
(c) detecting a first set of hybridization signals;
(d) treating the cell or tissue with a candidate drug;
(e) repeating steps (a)-(c) with a second nucleic acid sample from the treated cell or tissue and a second nucleic acid array identical to the first array to obtain a second set of hybridization signals; and
(f) comparing the first and second sets of hybridization signals, where any change in expression level of at least one protease gene, and/or one gene differentially expressed in osteoarthritic human cartilage cells as compared to osteoarthritis-free human cartilage cells, identifies the candidate drug as one that modulates expression of human protease or osteoarthritis genes.

In addition, the present invention features probe arrays for the detection of protein levels of human protease or osteoarthritis genes. Each of these probe arrays includes probes or probe sets that can specifically bind to protein products of respective human protease or osteoarthritis genes. Examples of human protease or osteoarthritis genes include, but are not limited to, those that encode the tiling sequences selected from Attachment C. In one embodiment, a probe array of the present invention comprises a plurality of antibodies, each of which can specifically bind to a protein product of a different respective human protease or osteoarthritis gene.

The present invention also features polynucleotide collections. In one embodiment, a polynucleotide collection of the present invention comprises a probe set capable of hybridizing under stringent or nucleic acid array hybridization conditions to a tiling sequence selected from Attachment C, or the complement thereof. In another embodiment, a polynucleotide collection of the present invention comprises at least 2, 5, 10, 100, 1,000, or more probe sets, each of which can hybridize under stringent or nucleic acid array hybridization conditions to a different respective tiling sequence selected from Attachment C, or the complement thereof. In yet another embodiment, a polynucleotide collection of the present invention includes at least 1, 2, 5, 10, 50, 100, 1,000 or more tiling sequences selected from Attachment C, or the complements thereof. In still another embodiment, a polynucleotide collection of the present invention comprises at least 1, 2, 5, 10, 100, 500, 1,000 or more sequences selected from SEQ ID NOs: 1-5,235, or the complements thereof.

Other features, objects, and advantages of the present invention are apparent in the detailed description that follows. It should be understood, however, that the detailed description, while indicating preferred embodiments of the invention, is given by way of illustration only, not limitation. Various changes and modifications within the scope of the invention will become apparent to those skilled in the art from the detailed description.

### BRIEF DESCRIPTION OF THE DRAWING

The drawing is provided for illustration, not limitation.

FIGURE 1 represents an Eisen cluster of transcriptional profiling data generated with a nucleic acid array of the present invention.

### DETAILED DESCRIPTION

I. Definitions

“Nucleic acid array hybridization conditions” refer to the temperature and ionic conditions that are normally used in nucleic acid array hybridization. These conditions include 16-hour hybridization at 45° C., followed by at least three 10-minute washes at room temperature. The hybridization buffer comprises 100 mM MES, 1 M [Na⁺], 20 mM EDTA, and 0.01% Tween 20. The pH of the hybridization buffer preferably is between 6.5 and 6.7. The wash buffer is 6×SSPET. 6×SSPET contains 0.9 M NaCl, 60 mM NaH₂PO₄, 6 mM EDTA, and 0.005% Triton X-100. Under more stringent nucleic acid array hybridization conditions, the wash buffer can contain 100 mM MES, 0.1 M [Na⁺], and 0.01% Tween 20.

“A substantial portion of all polynucleotide probes” means at least 15% of all polynucleotide probes. For instance, a substantial portion can be at least 20%, 25%, 30%, 35%, 40%, 45%, 50%, or more of all polynucleotide probes. Where a nucleic acid array includes both perfect match probes and perfect mismatch probes, a substantial portion of all polynucleotide probes can include, for example, at least 30% of all perfect match probes. Preferably, a substantial portion of all polynucleotide probes includes at least 50%, 60%, 70%, 80%, 90% or more of all perfect match probes.

The expression level of a gene is “substantially higher” in one tissue than in another tissue if the molar concentration of the mRNA transcript of the gene relative to the total mRNA in the former tissue is at least 1.5-fold of that in the latter tissue. For instance, the molar concentration of the mRNA transcript of the gene relative to the total mRNA in the former tissue can be at least 2-fold, 5-fold, 10-fold, or 20-fold of that in the latter tissue. In one instance, the mRNA transcript of the gene is detectable in the former tissue but not in the latter tissue. In another instance, the mRNA transcript of the gene is more readily identifiable using 5′ or 3′ sequence reads from a cDNA library prepared from the former tissue than from a cDNA library prepared from the latter tissue.

“Stringent conditions” are at least as stringent as, for example, conditions G-L shown in Table 1. In certain embodiments of the present invention, highly stringent conditions A-F can be used. Under Table 1, hybridization is carried out under the hybridization conditions (Hybridization Temperature and Buffer) for about four hours, followed by two 20-minute washes under the corresponding wash conditions (Wash Temp. and Buffer).

TABLE 1
Stringency Conditions

| | Poly- | | | |
|---|---|---|---|---|
| Stringency | nucleotide | Hybrid | Hybridization | Wash Temp. |
| Condition | Hybrid | Length (bp)¹ | Temperature and Buffer^{H} | and Buffer^{H} |
| A | DNA:DNA | >50 | 65° C.; 1 × SSC -or- | 65° C.; 0.3 × SSC |
| | | | 42° C.; 1 × SSC, 50% formamide | |
| B | DNA:DNA | <50 | T_{B}*; 1 × SSC | T_{B}*; 1 × SSC |
| C | DNA:RNA | >50 | 67° C.; 1 × SSC -or- | 67° C.; 0.3 × SSC |
| | | | 45° C.; 1 × SSC, 50% formamide | |
| D | DNA:RNA | <50 | T_{D}*; 1 × SSC | T_{D}*; 1 × SSC |
| E | RNA:RNA | >50 | 70° C.; 1 × SSC -or- | 70° C.; 0.3 × SSC |
| | | | 50° C.; 1 × SSC, 50% formamide | |
| F | RNA:RNA | <50 | T_{F}*; 1 × SSC | T_{f}*; 1 × SSC |
| G | DNA:DNA | >50 | 65° C.; 4 × SSC -or- | 65° C.; 1 × SSC |
| | | | 42° C.; 4 × SSC, 50% formamide | |
| H | DNA:DNA | <50 | T_{H}*; 4 × SSC | T_{H}*; 4 × SSC |
| I | DNA:RNA | >50 | 67° C.; 4 × SSC -or- | 67° C.; 1 × SSC |
| | | | 45° C.; 4 × SSC, 50% formamide | |
| J | DNA:RNA | <50 | T_{J}*; 4 × SSC | T_{J}*; 4 × SSC |
| K | RNA:RNA | >50 | 70° C.; 4 × SSC -or- | 67° C.; 1 × SSC |
| | | | 50° C.; 4 × SSC, 50% formamide | |
| L | RNA:RNA | <50 | T_{L}*; 2 × SSC | T_{L}*; 2 × SSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the | | | | |
| # polynucleotides and identifying the region or regions of optimal sequence complementarity. | | | | |
| ^{H}SSPE (1 × SSPE is 0.15 M NaCl, 10 mM NaH₂PO₄, and 1.25 mM EDTA, pH 7.4) can be substituted for SSC (1 × SSC is 0.15 M NaCl and 15 mM sodium citrate) in the hybridization and wash buffers. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, | | | | |
| # Tₘ(° C.) = 2(# of A + T bases) + 4(# of G + C bases). For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the molar concentration of sodium ions | | | | |
| # in the hybridization buffer (Na⁺ for 1 × SSC = 0.165 M). | | | | |

Various aspects of the invention are described in further detail in the following sections or subsections. The use of sections and subsections is not meant to limit the invention; each section and subsection may apply to any aspect of the invention.

II. The Invention

The nucleic acid arrays of the present invention comprise polynucleotide probes for human protease genes and/or human osteoarthritis genes. The osteoarthritis genes are differentially expressed in osteoarthritic human cartilage cells as compared to osteoarthritis-free human cartilage cells. The probes for human protease and/or osteoarthritis genes can hybridize under stringent or nucleic acid array hybridization conditions to the mRNA and/or cDNA sequences of these genes, or the complements thereof. In one embodiment, a nucleic acid array of the present invention includes one or more substrate supports, and a substantial portion of all polynucleotide probes that are stably attached to the one or more substrate supports consists of probes for human protease and/or osteoarthritis genes. In another embodiment, a nucleic acid array of the present invention includes probes which can hybridize under stringent or nucleic acid array hybridization conditions to respective tiling sequences selected from Attachment C, or the complements thereof. The nucleic acid array can also include probes for other genes that are not associated with human osteoarthritis or proteases.

The nucleic acid arrays of the present invention can be used to detect or monitor the expression profiles of human protease and/or osteoarthritis genes. The nucleic acid arrays of the present invention can also be used to identify new therapeutic targets for the treatment of osteoarthritis and/or protease-related diseases. In addition, the nucleic acid arrays of the present invention can be used to screen for potential drug candidates for treating osteoarthritis and/or protease-related diseases.

Compared to a typical Affymetrix microarray, the nucleic acid arrays of the present invention are concentrated with probes for human protease and/or osteoarthritis genes. This allows for a more focused, cost-effective study on osteoarthritis and other protease-related diseases. For instance, a typical Affymetrix microarray, such as the Human Genome U133 Set, includes probe sets for approximately 33,000 human genes. The gene expression analysis using this microarray may generate hundreds, if not thousands, of genes which have altered expression in response to an osteoarthritis treatment. Interpretation of this gene expression data is frequently laborious and time-consuming because many of the genes with altered expression are not associated with osteoarthritis. Moreover, microarrays with comprehensive representation of all human genes are expensive, therefore preventing their widespread use in the drug development process. By concentrating probes for human protease and/or osteoarthritis genes on a single array, the present invention eliminates the painstaking process for identifying and removing irrelevant genes. In addition, by using a less number of probes, the present invention reduces the cost associated with the use of traditional arrays, thereby accelerating the drug development process.

A. Collection of mRNA, cDNA and/or Other Polypeptide Coding Sequences of Human Protease Genes and Human Osteoarthritis Genes

mRNA, cDNA and/or other polypeptide coding sequences of human protease genes can be collected from a variety of sources, such as GenBank and TIGR (The Institute for Genome Research). These publicly accessible sequence databases frequently include a large number of EST and cDNA sequences. Many of these sequences are annotated. Sequences encoding human proteases can therefore be identified.

The publicly available sequence databases also contain an enormous amount of human genomic sequences. Open reading frames (ORFs) in these genomic sequences can be predicted or isolated using methods known in the art. Suitable methods for this purpose include, but are not limited to, GeneMark (provided by the European Bioinformatics Institute), Glimmer (provided by TIGR), and ORF Finder (provided by the National Center for Biotechnology Information (NCBI)). The ORFs that encode or have high sequence homology to known proteases can be identified. The function of the polypeptides encoded by the identified ORFs can be further evaluated using standard methods, such as in vitro transcription and translation or the cell culture-based assays.

Uncontrolled protease activity has been implicated in osteoarthritis and many other diseases, such as arteriosclerosis, muscular dystrophy, amyotrophy, rheumatoid arthritis, autoimmune diseases, inflammation, infection, cancer, and degenerative disorders. Therefore, proteases have been the major targets for drug action and development.

Proteases are known to be involved in a wide variety of biological processes, including post-translational modifications, blood coagulation, fibrinolysis, complement activation, fertilization, hormone production, degradation of undesirable proteins and invading organisms, tumor metastasis, stress response, wound healing, tissue remodeling, cell proliferation/differentiation, and signal transduction pathways. Protease includes endopeptidases and exopeptidases. Endopeptidases cleave peptide bonds at points within the protein, while exopeptidases remove amino acids sequentially from either N or C-terminus. At least four mechanistic classes of endopeptidases have been recognized: the aspartic, the serine, the metallo, and the cysteine proteinases.

The aspartic proteinases include at least one active aspartate residue at the catalytic center. Catalysis by aspartic proteases involves the formation of a non-covalent neutral tetrahedral intermediate. Examples of the aspartic proteases include pepsin A, presenilin 1, chymosin, lysosomal cathepsins D, renin, and retropepsin (from human immunodeficiency virus type 1).

The serine proteinases are a large family of proteolytic enzymes, including trypases (cleaving arginine or lysine), aspases (cleaving after aspartate), chymases (cleaving after phenylalanine or leucine), metases (cleaving after methionine), and serases (cleaving after serine). The serine proteases are so named because of the presence of a serine residue in the active catalytic site of the protease.

The metallo proteinases differ widely in their sequences and their structures. Many of the metallo proteinases contain a zinc atom in their catalytic sites. Examples of the metallo proteinases include membrane alanyl aminopeptidase, germinal peptidyl-dipeptidase A, collagenase 1, neprilysin, carboxypeptidase A, membrane dipeptidase, and S2P protease.

The cysteine proteinases contain a cysteine nucleophile at the catalytic site. Like the serine proteinases, catalysis by cysteine proteinases involves the formation of a covalent intermediate between the substrate and the active-site cysteine. Exemplary cysteine proteinases include cytosolic calpains and lysosomal cathepsins.

The present invention is not limited to proteases that are known to be involved in osteoarthritis. Other protease genes and their mRNA/cDNA sequences can also be identified and used to prepare probes for constructing the nucleic acid arrays of the present invention.

mRNA, cDNA and/or other polypeptide-coding sequences of human osteoarthritis genes can be obtained through sequencing suitable cDNA libraries. Exemplary cDNA libraries for this purpose include libraries prepared from osteoarthritic human cartilage cells and libraries prepared from osteoarthritis-free human cartilage cells. Preferably, each library is constructed such that the frequency of occurrence of each cDNA clone in the cDNA library is proportional to the relative molar concentration of the corresponding mRNA in the cartilage tissue from which the cDNA library is derived. The frequency of occurrence of each cDNA clone also correlates with the chance of that cDNA clone being detected in the cDNA library. Thus, the readiness of a cDNA clone being detected in the cDNA library can reflect the relative concentration of the corresponding mRNA in the cartilage tissue from which the cDNA library is derived. Accordingly, by comparing the sequence reads obtained from osteoarthritic cDNA libraries to those obtained from osteoarthritis-free libraries, genes that are differentially expressed in these two types of libraries may be determined.

Methods for constructing cartilage cDNA libraries are well known in the art. Suitable cartilage tissues for this purpose include hyaline cartilage, elastic cartilage, fibrous cartilage, and articular cartilage. Preferably, the cartilage tissues are isolated from the large joints of osteoarthritis-free humans or humans who are affected with osteoarthritis. In one embodiment, osteoarthritic cartilage is obtained from osteoarthritis patients undergoing total knee arthroplasty, and osteoarthritic-free cartilage is collected from the femoral heads of osteoarthritic-free patients who undergo hemiarthroplasty for hip facture. The isolated cartilage samples can be homogenized and then extracted for mRNA. Suitable agents for mRNA extraction include, but are not limited to, guanidine isothiocyanate/acidic phenyl method, the TRIZOL® Reagent (Invitrogen), or the Micro-FastTrack™ 2.0 or FastTrack™ 2.0 mRNA Isolation Kits (Invitrogen). Alternatively, cartilage cells, such as chondrocytes, can be first dissociated from the cartilage samples, and then extracted for mRNA. The extracted mRNA is subsequently purified based on its unique 3′ or 5′ structure.

In one embodiment, the mRNA in the cartilage cells is purified by virtue of the presence of a polyadenylated (polyA) tail present at the 3′ end of the mRNA. The polyA tail binds to a resin conjugated with oligo-dT (oligo-dT chromatography). The purified mRNA is then copied into cDNA using a reverse transcriptase and a primer under conditions sufficient for the first strand cDNA synthesis to occur. Although both random and specific primers can be employed, in many embodiments the primer is an oligo-dT primer that provides for hybridization to the polyA tail in the mRNA. The oligo-dT primer is sufficiently long to provide for efficient hybridization to the polyA tail. Typically, the oligo-dT primer ranges from 10 to 25 nucleotides in length, such as from 12 to 18 nucleotides in length. Additional reagents, such as dNTPs, buffering agents (e.g. Tris Cl), cationic sources (monovalent or divalent, e.g. KCl, MgCl₂), and sulfhydril reagents (e.g. dithiothreitol), can be included in the reaction.

A variety of enzymes, usually DNA polymerases possessing reverse transcriptase activity, can be used for the first strand cDNA synthesis. Examples of suitable DNA polymerases include the DNA polymerases derived from thermophilic bacteria, archaebacteria, retroviruses, yeasts, Neurosporas, Drosophilas, primates, or rodents. In one embodiment, the DNA polymerase is derived from Moloney murine leukemia virus (M-MLV), human T-cell leukemia virus type I (HTLV-I), bovine leukemia virus (BLV), Rous sarcoma virus (RSV), human immunodeficiency virus (HIV), Thermus aquaticus (Taq), Thermus thermophilus (Tth), or avian reverse transcriptase. M-MLV reverse transcriptase lacking RNaseH activity can also be used. See, for example, U.S. Pat. No. 5,405,776, which is incorporated herein by reference.

The order in which the reagents are combined can be modified as desired. In one protocol, all reagents except for the reverse transcriptase are combined on ice, and then the reverse transcriptase is added at around 4° C. Following the addition of the reverse transcriptase, the temperature of the reaction mixture can be raised to 37° C., followed by incubation for a period of time sufficient for the primer extension to form the first strand of cDNA. The primer extension starts at the 3′ end of the mRNA and proceeds towards the 5′ end. The incubation period can take about 1 hour.

Second strand cDNA synthesis is then performed. Linkers are added to the ends of the double stranded cDNA to allow for its package into virus or cloning into plasmids/vectors. At this stage, the cDNA is in a form that can be propagated. The linkers or the primers can include rare restriction enzyme sites, such as Not I and/or Pac I, to facilitate the cloning of the cDNA into plasmids/vectors. Suitable plasmids/vectors for subcloning cDNA molecules include, but are not limited to, the pT7T3-Pac vector (a modified pT7T3 vector, Pharmacia), the pSPORT 1 vector (Invitrogen), and the lambda vectors (Stratagene).

In another embodiment, the mRNA in the cartilage cells is purified through its unique 5′-cap structure. The 5′-cap structure of eukaryotic mRNA includes m7 GpppN, where N can be any nucleotide. Resins conjugated with a 5′-cap binding agent can be used to purify mRNA. Suitable 5′-cap binding agents include, but are not limited to, the eIF-4E/eIF-4G fusion protein disclosed in U.S. Pat. No. 6,326,175, which is incorporated herein by reference. The first strand cDNA synthesis can be performed using any conventional protocol. Following the first strand cDNA synthesis, the resultant mRNA/DNA duplex is contacted with an RNase to degrade single stranded RNA but not RNA complexed to DNA. Suitable RNases for this purpose include RNase Ti from Aspergillus orzyae, RNase I, and RNase A. The conditions and duration of incubation during this step can vary depending on the specific nuclease employed. Generally, the incubation temperature is between about 20° C. to 37° C., and the incubation time lasts from about 10 to 60 min.

Nuclease treatment produces blunt-ended mRNA/DNA duplexes. The mRNA/DNA hybrids that include the unique 5′-cap structure can be isolated using resins conjugated with the eIF-4E/eIF-4G fusion protein. Following isolation, the nucleic acids can be further processed, including release from the resins and production of double stranded cDNA. The double stranded cDNA is then subcloned into appropriate plasmids/vectors to create a cDNA library.

In one specific example, the cDNA library is prepared using the CloneMiner™ cDNA Library Construction Kit provided by Invitrogen (Carlsbad, Calif.). The CloneMiner Kit uses a modified reverse transcriptase and a biotin-attB-oligo(dT) primer to synthesize the first strand of cDNA. The modified reverse transcriptase has reduced RNAase H activity, thereby decreasing RNA degradation during the first strand synthesis. The second strand of cDNA is synthesized using E. coli DNA polymerase I, and an attB adaptor is added to the 5′ end of the double stranded cDNA. The final cDNA product is therefore flanked by two attB sites.

The att sites, such as the attB and attP sites, are components of the lambda recombination system. Recombination between the attB and attP sites swaps the sequences located therebetween. The CloneMiner destination vectors contain the ccdB gene flanked by the attP sites. The ccdB gene inhibits the growth of most E. coli strains. Recombination between the attB-flanked cDNA sequence and the destination vectors replaces the ccdB gene with the cDNA sequence, thereby removing the inhibitory effect of the ccdB gene and allowing negative selection of the recombinant vector that contains the cDNA insert. The selected recombinant vectors are then transformed into competent E. coli cells to produce a cDNA library. The cDNA library prepared using the CloneMiner cDNA Library Construction Kit preferably includes at least 5×10⁶, 1×10⁷, 5×10⁷ or more primary clones.

According to the CloneMiner user's manual, cDNA can be either radiolabeled or non-radiolabeled during its synthesis. Radiolabeling facilitates the measurement of cDNA yield and overall quality of the first strand cDNA synthesis. For instance, if [α-³²P]dCTP is used to monitor the first strand reaction, the percent incorporation of [α-³²P]dCTP preferably is no less than 10%. More preferably, the percent incorporation of [α-³²P]dCTP is about 20-50%.

In addition, cDNA can be size fractionated before being subcloned into the destination vectors. Suitable methods for size fractionation include, but are not limited to, column chromatography and gel electrophoresis. The final cDNA yield after size fractionation and subsequent ethanol precipitation preferably is no less than 30-40 ng. In some cases, at least 50, 75, 100, 150, 200 ng or more cDNA is used for subcloning.

During the construction of the cDNA library, the mRNA extraction and purification steps are preferably conducted under conditions where the RNase activities are minimized. The quality of the purified mRNA can be monitored using agarose/ethidium bromide gel electrophoresis. The amount of the purified mRNA can range from 0.5 to 10 μg. Preferably, at least 2 μg of purified mRNA is used for the construction of a cDNA library. In one embodiment, 1 to 5 μg of mRNA is used for preparing a cDNA library containing 10⁶ to 10⁷ primary clones in E. coli.

cDNA clones in a cartilage library can be readily sequenced using methods known in the art. In standard methods, individual cDNA clones in the library are first isolated, followed by the purification of vectors that contain the cDNA inserts. The cDNA inserts can then be sequenced using primers designed from the common vector sequences adjacent to the 5′ or 3′ end of the cDNA inserts.

In one embodiment, the 5′ and 3′ sequence reads from an osteoarthritic cartilage library as well as an osteoarthritis-free cartilage library are collected. Both libraries are prepared using oligo-dT primers for the first strand cDNA synthesis. The frequency of occurrence of each cDNA clone in each library is proportional to the relative molar concentration of the corresponding mRNA in the cartilage tissue from which the cDNA library is derived. Therefore, the readiness of a cDNA clone being detected in the cDNA library may represent the relative abundance of the corresponding mRNA in the cartilage tissue from which the library is derived.

The 5′ and 3′ sequence reads from the cartilage libraries can be edited before being used for other purposes. For instance, the vector sequences at the 5′ end of the 3′ sequence read product can be removed or masked out. This process may be carried out automatically, such as by employing a screening algorithm, or conducted manually. Typically, the quality of the sequence read decreases as it moves towards the distant end of synthesis. Thus, by trimming the distant end, the overall quality and accuracy of the eventual sequence will be improved. In addition to trimming the distant end, the initiation end of synthesis for each sequence read can also be trimmed.

In a preferred embodiment, the 5′ sequence reads are first mapped to publicly available human gene sequences (such as those from GenBank or NCBI's human RefSeq). These publicly available gene sequences are then used in the nucleic acid array design process as described below. If a 5′ sequence read does not map to any known human gene in the public sequence databases, the 5′ sequence read can be resubmitted for 3′ sequencing.

The edited 3′ sequence reads from both the osteoarthritic cartilage library and the osteoarthritis-free cartilage library, the sequences derived from the 5′ sequence reads, and the protease sequences obtained from GenBank and other sequence databases, can be clustered to identify highly homologous sequences. Suitable clustering algorithms for this purpose include, but are not limited to, the CAT (cluster and alignment tool) software package provided by DoubleTwist. See Clustering and Alignment Tools User's Guide (DoubleTwist, Inc., 2000).

The CAT program can reduce the redundancy, as well as mask low-complexity regions of the input sequence set. The resulting sequence set derived from CAT contains two distinct groups of sequences. The first group is a set of consensus sequences derived from multiple sequence alignment produced for CAT sub-clusters containing more than one sequence. These multi-sequence sub-clusters may include single transcripts represented in the input sequence set numerous times. The second group is a set of exemplar sequences that do not cluster with any other CAT sub-cluster. The consensus and exemplar sequences can be generated such that any base ambiguity would be identified with the respective IUPAC (International Union of Pure and Applied Chemistry) base representation, which is identical to the WIPO Standard ST.25 (1998).

In a small number of cases, the multi-sequence sub-clusters contain a large number of sequences due to clustering artifacts (e.g., highly homologous genes or domains). In these cases, through more stringent clustering parameters, the large sub-clusters are re-clustered. In addition, the consensus sequences can be manually curated to verify cluster membership.

In a specific example, a set of 47,600 sequences were collected from NCBI's human RefSeq collection. With this set of RefSeq sequences, 5,553 5′ sequence reads from a mild osteoarthritic cartilage cDNA library (“GI_MILD”), 5,332 5′ sequence reads from a severe osteoarthritic cartilage cDNA library (“GI_SEVERE”), as well as 5,224 5′ sequences reads from an osteoarthritis-free cartilage library (“GLAXO_Normal”) and 5,019 5′ sequences reads from an osteoarthritic cartilage library (“GLAXO_OA”), were clustered and aligned to determine which known genes were present in the cartilage libraries. From this 5′ clustering run, most of the 5′ sequence reads were mapped to a known gene. Those that did not map to a known gene were resubmitted for 3′ sequencing. These 3′ sequences were then clustered. The combination of these two cluster collections in addition to a list of known proteases were used to generate probes for constructing nucleic acid arrays.

Examples of the consensus sequences obtained using the above-described method are illustrated in Attachment A. Examples of the exemplar sequences are shown in Attachment B. Each consensus or exemplar sequence has a respective SEQ ID NO and a header that includes the qualifier (starting with “wyeHumanOAla”) and other information of that sequence. The consensus and exemplar sequences are collectively referred to as the “parent sequences.”

Attachment F illustrates the source(s) from which each parent sequence is derived. If at least one input sequence for a parent sequence is from the osteoarthritis-free cartilage cDNA library, then the “GLAXO_Normal” column for that parent sequence is selected as “1.” Otherwise, “GLAXO_Normal” is “0.” Likewise, if at least one input sequence for a parent sequence is from the osteoarthritic cartilage library, the mild osteoarthritic cartilage cDNA library, or the severe osteoarthritic cartilage cDNA library, then “GLAXO_OA,” “GI_MILD,” or “GI_SEVERE” for that parent sequence is selected as “1,” respectively. Otherwise, “GLAXO_OA” “GI_MILD,” and “GI_SEVERE” are “0.” If at least one input sequence is derived from a source (such as NCBI's human RefSeq) other than the above-described four cartilage cDNA libraries, then the “Other” column for the parent sequence is “1.” Occasionally, if an input sequence cannot be determinably assigned to either “GI_MILD,” or “GI_SEVERE,” the “Other” column for the parent sequence is selected as “1.”

In one example, all the input sequences for a parent sequence are derived from the osteoarthritis-free cartilage library and/or non-cDNA library sources (“GLAXO_Normal” or “Other” being “1”). These input sequences were detectable in the osteoarthritis-free cartilage library, but not in the osteoarthritic cartilage libraries. As discussed above, the chance for a sequence being detected in a cDNA library generally correlates with the relative concentration of the corresponding mRNA in the tissue from which the library is derived. Accordingly, the parent sequence can represent an mRNA transcript or a gene whose level of expression in the osteoarthritis-free cartilage tissue is substantially higher than that in the osteoarthritic cartilage tissue. For instance, the level of expression of the mRNA transcript or gene in the osteoarthritis-free cartilage tissue can be at least 1.5-fold, 2-fold, 3-fold, 4-fold, or 5-fold of that in the osteoarthritic cartilage tissue. The level of expression can be determined using standard methods, such as RT-PCR, Northern Blot, microarrays, or immunoassays such as ELISA or RIA.

In another specific example, all the input sequences for a parent sequence are derived from one of the osteoarthritic cartilage cDNA libraries (“GLAXO_OA,” “GI_MILD,” or “GI_SEVERE” being “1”). These input sequences were detectable in the osteoarthritic cartilage libraries, but not in the osteoarthritis-free cartilage library. The parent sequence therefore can represent an mRNA transcript or a gene whose level of expression in the osteoarthritic cartilage tissue is substantially higher than that in the osteoarthritis-free cartilage tissue.

In a further example, the input sequences for a parent sequence are derived from both the osteoarthritic cartilage cDNA libraries and the osteoarthritis-free cartilage library. The parent sequence can represent an mRNA transcript or a gene whose level of expression in the osteoarthritic cartilage tissue is substantially the same as that in the osteoarthritis-free cartilage tissue.

In yet another specific example, all the input sequences for a parent sequence are derived from non-cDNA library sources such as NCBI's human Refseq (“Other” being 1).

In still yet another specific example, the input sequences for a parent sequence are detectable in the severe osteoarthritic cartilage library, but not in the mild osteoarthritic cartilage library, or vice versa. This suggests that the parent sequence can be differentially expressed in severely affected cartilage tissues as compared to mildly affected cartilage tissues.

B. Preparation of Polynucleotide Probes for Expression Profiling of Human Protease or Osteoarthritis Genes

The consensus and exemplar sequences depicted in Attachments A and B can be used to prepare polynucleotide probes that are useful for expression profiling of human protease or osteoarthritis genes. The polynucleotide probes for each parent sequence can hybridize under stringent or nucleic acid array hybridization conditions to that parent sequence, or the complement thereof. Preferably, the probes for each parent sequence are incapable of hybridizing under stringent or nucleic acid array hybridization conditions to other parent sequences, or the complements thereof. If a parent sequence contains one or more ambiguous residues, the probes for that parent sequence can hybridize under stringent or nucleic acid array hybridization conditions to the longest unambiguous segment of that parent sequence, or the complement thereof. In one embodiment, the probe for a parent sequence comprises or consists of an unambiguous sequence fragment of that parent sequence, or the complement thereof.

The length of each polynucleotide probe can be selected to produce the desired hybridization effects. For example, the probes can include or consist of at least 10, 15, 20, 25, 30, 35, 40, 45, 50, 60, 70, 80, 90, 100, 200, 300, 400 or more consecutive nucleotides. The probes can be DNA, RNA, or PNA. Other modified forms of DNA, RNA, or PNA can also be used. The nucleotide units in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

In one embodiment, the probes have relatively high sequence complexity, and preferably do not contain long stretches of the same nucleotide. In another embodiment, the probes can be designed such that they do not have a high proportion of G or C residues at the 3′ ends. In yet another embodiment, the probes do not have a 3′ terminal T residue. Depending on the type of assay or detection to be performed, sequences that are predicted to form hairpins or interstrand structures, such as “primer dimers,” can be either included in or excluded from the probe sequences. Preferably, each probe does not contain any ambiguous base.

Any part of a parent sequence can be used to prepare probes. For instance, probes can be prepared from the protein-coding region, the 5′ untranslated region, or the 3′ untranslated region of a parent sequence. Multiple probes, such as 5, 10, 15, 20, 25, 30, or more, can be prepared for each parent sequence. The multiple probes for the same parent sequence may or may not overlap each other, although overlap among different probes may be desirable in some assays.

In a preferred embodiment, the probes for a parent sequence have low sequence identities with other parent sequences, or the complements thereof. For instance, each probe for a parent sequence can have no more than 70%, 60%, 50% or less sequence identity with other parent sequences, or the complements thereof. This reduces the risk of potential cross-hybridization between the probes and the undesirable RNA transcripts. Sequence identity can be determined using methods known in the art. These methods include, but are not limited to, BLASTN, FASTA, FASTDB, and the GCG program.

The suitability of the probes for hybridization can be evaluated using various computer programs. Suitable programs for this purpose include, but are not limited to, LaserGene (DNAStar), Oligo (National Biosciences, Inc.), MacVector (Kodak/IBI), and the standard programs provided by the Genetics Computer Group (GCG).

The polynucleotide probes of the present invention can be synthesized using methods known in the art. Exemplary methods include automated or high throughput DNA synthesizers, such as those provided by Millipore, GeneMachines, and BioAutomation. Preferably, the synthesized probes are substantially free of impurities, such as incomplete products produced during the synthesis. In addition, the probes are substantially free of other contaminants that may hinder the desired functions of the probes. The probes can be purified or concentrated using different methods, such as reverse phase chromatography, ethanol precipitation, gel filtration, electrophoresis, or any combination thereof.

In one embodiment, the parent sequences with large sizes are divided into shorter sequence segments to facilitate the probe design. These divided sequences, together with the undivided parent sequences, are collectively referred to as the “tiling sequences.”

Attachment C depicts the tiling sequences and their respective headers. The headers include the qualifiers (starting with “wyeHumanOAla”) and other information of the tiling sequences. The first 321 tiling sequences in Attachment C correspond to, in consecutive order, the consensus sequences in Attachment A. The remaining tiling sequences correspond to, in consecutive order, the exemplar sequences in Attachment B.

Attachment D shows the location of each tiling sequence in the corresponding parent sequence. The 5′ end of each tiling sequence in the corresponding parent sequence is indicated under “TilingStart,” and the 3′ end of the tiling sequence is shown under “TilingEnd.”

Polynucleotide probes for each tiling sequence can hybridize under stringent or nucleic acid array hybridization conditions to that tiling sequence, or the complement thereof. Preferably, a probe for a tiling sequence can hybridize under highly stringent conditions to the tiling sequence, or the complement thereof. More preferably, the probes for a tiling sequence are incapable of hybridizing under stringent or nucleic acid array hybridization conditions to other tiling sequences, or the complements thereof. If a tiling sequence contains one or more ambiguous residues, the probes for the tiling sequence can hybridize under stringent or nucleic acid array hybridization conditions to the longest unambiguous segment of that sequence, or the complement thereof.

Any suitable method can be used to prepare probes for the tiling sequences. In one embodiment, the probes are generated using Array Designer, a software package provided by TeleChem International, Inc (Sunnyvale, Calif. 94089). Examples of the probes thus generated are illustrated in Attachment E. The location of the 5′ and 3′ ends of each probe in the corresponding tiling sequence is shown under “5′ End” and “3′ End,” respectively. Other methods or software programs can also be used to generate hybridization probes for the tiling sequences.

The parent sequences, tiling sequences, and polynucleotide probes of the present invention can be used to detect or monitor the expression profiles of human protease or osteoarthritis genes. Methods suitable for this purpose include, but are not limited to, nucleic acid arrays (including bead arrays), Southern Blot, Northern Blot, PCR, and RT-PCR. The expression profiles of other genes that are expressed in human cartilage tissues can also be evaluated using the present invention.

C. Nucleic Acid Arrays for Detecting Expression Profiles of Human Protease or Osteoarthritis Genes

The polynucleotide probes of the present invention can be used to make nucleic acid arrays. A typical nucleic acid array includes at least one substrate support. The substrate support includes a plurality of discrete regions. The location of each discrete region is either known or determinable. The discrete regions can be organized in various forms or patterns. For instance, the discrete regions can be arranged as an array of regularly spaced areas on the surface of the substrate. Other patterns, such as linear, concentric or spiral patterns, can be used. In one embodiment, a nucleic acid array of the present invention is a bead array which includes a plurality of beads stably associated with the polynucleotide probes of the present invention.

Polynucleotide probes can be stably attached to their respective discrete regions through covalent and/or non-covalent interactions. By “stably attached” or “stably associated,” it means that during nucleic acid array hybridization the polynucleotide probe maintains its position relative to the discrete region to which the probe is attached. Any suitable method can be used to attach polynucleotide probes to a nucleic acid array substrate. In one embodiment, the attachment is achieved by first depositing the polynucleotide probes to their respective discrete regions and then exposing the surface to a solution of a cross-linking agent, such as glutaraldehyde, borohydride, or other bifunctional agents. In another embodiment, the polynucleotide probes are covalently bound to the substrate via an alkylamino-linker group or by coating the glass slides with polyethylenimine followed by activation with cyanuric chloride for coupling the polynucleotides. In yet another embodiment, the polynucleotide probes are covalently attached to a nucleic acid array through polymer linkers. The polymer linkers may improve the accessibility of the probes to their purported targets. Preferably, the polymer linkers are not involved in the interactions between the probes and their purported targets.

In addition, the polynucleotide probes can be stably attached to a nucleic acid array substrate through non-covalent interactions. In one embodiment, the polynucleotide probes are attached to the substrate through electrostatic interactions between positively charged surface groups and the negatively charged probes. In another embodiment, the substrate is a glass slide having a coating of a polycationic polymer on its surface, such as a cationic polypeptide. The probes are bound to these polycationic polymers. In yet another embodiment, the methods described in U.S. Pat. No. 6,440,723, which is incorporated herein by reference, are used to attach the probes to the nucleic acid array substrate(s).

Various materials can be used to make the substrate support. Suitable materials include, but are not limited to, glasses, silica, ceramics, nylons, quartz wafers, gels, metals, and papers. The substrates can be flexible or rigid. In one embodiment, they are in the form of a tape that is wound up on a reel or cassette. Two or more substrate supports can be used in the same nucleic acid array. Preferably, the substrate is non-reactive with reagents that are used in nucleic acid array hybridization.

The surfaces of the substrate support can be smooth and substantially planar. The surfaces of the substrate can also have a variety of configurations, such as raised or depressed regions, trenches, v-grooves, mesa structures, and other irregularities. The surfaces of the substrate can be coated with one or more modification layers. Suitable modification layers include inorganic and organic layers, such as metals, metal oxides, polymers, or small organic molecules. In one embodiment, the surface(s) of the substrate is chemically treated to include groups such as hydroxyl, carboxyl, amine, aldehyde, or sulfhydryl groups.

The discrete regions on the substrate can be of any size, shape and density. For instance, they can be squares, ellipsoids, rectangles, triangles, circles, other regular or irregular geometric shapes, or any portion or combination thereof. In one embodiment, each of the discrete regions has a surface area of less than 10⁻¹ cm², such as less than 10⁻², 10⁻³, 10⁻⁴, 10⁻⁵, 10⁻⁶, or 10⁻⁷ cm². In another embodiment, the spacing between each discrete region and its closest neighbor, measured from center-to-center, is in the range of from about 10 to about 400 μm. The density of the discrete regions may range, for example, between 50 and 50,000 regions/cm².

All of the methods known in the art can be used to make the nucleic acid arrays of the present invention. For instance, the probes can be synthesized in a step-by-step manner on the substrate, or can be attached to the substrate in pre-synthesized forms. Algorithms for reducing the number of synthesis cycles can be used. In one embodiment, a nucleic acid array of the present invention is synthesized in a combinational fashion by delivering monomers to the discrete regions through mechanically constrained flowpaths. In another embodiment, a nucleic acid array of the present invention is synthesized by spotting monomer reagents onto a substrate support using an ink jet printer (such as the DeskWriter C manufactured by Hewlett-Packard). In yet another embodiment, polynucleotide probes are immobilized on a nucleic acid array of the present invention by using photolithography techniques.

The nucleic acid arrays of the present invention can also be bead arrays which comprise a plurality of beads. Polynucleotide probes can be stably attached to each bead using any of the above-described methods.

In one embodiment, a substantial portion of all polynucleotide probes on a nucleic acid array of the present invention can hybridize under stringent or nucleic acid array hybridization conditions to human protease genes or human osteoarthritis genes. In one specific example, at least 25%, 35%, 45%, 50%, or more of all polynucleotide probes on the nucleic acid array can hybridize to human protease or osteoarthritis genes. The probes for these human genes can be concentrated on one substrate support. They can also be attached to two or more substrate supports, such as in the bead arrays.

Any number of polynucleotide probes can be included in a nucleic acid array of the present invention. For instance, the nucleic acid array can include at least 2, 5, 10, 20, 30, 40, 50, 100, 200, 300, 400, 500, 1,000 or more different probes, and each probe can hybridize under stringent or nucleic acid array hybridization conditions to a different respective gene selected from human protease genes and human osteoarthritis genes. In one embodiment, a nucleic acid array of the present invention includes a first set of probes which are capable of hybridizing under stringent or nucleic acid array hybridization conditions to different respective human osteoarthritis genes. The expression level of each of these osteoarthritis genes is substantially higher (such as at least 1.5-fold, 2-fold, 5-fold, or greater) in osteoarthritic human cartilage cells than in osteoarthritis-free human cartilage cells. In another embodiment, a nucleic acid array of the present invention includes a second set of probes which are capable of hybridizing under stringent or nucleic acid array hybridization conditions to different respective human osteoarthritis genes, and the expression levels of these osteoarthritis are substantially higher in osteoarthritis-free human cartilage cells than in osteoarthritic human cartilage cells. In yet another embodiment, a nucleic acid array of the present invention includes a third set of probes which are capable of hybridizing under stringent or nucleic acid array hybridization conditions to different respective human protease genes. Each of the above-described probe sets can include at least 2, 5, 10, 50, 100, 200, 300, 400, 500, or more different probes.

In yet another embodiment, a nucleic acid array of the present invention includes at least 2, 5, 10, 20, 30, 40, 50, 100, 200, 300, 400, 500, 1,000, 2,000, 3,000, 4,000, 5,000, or more different probes, and each probe can hybridize under stringent or nucleic acid array hybridization conditions to a different respective tiling sequence selected from Attachment C, or the complement thereof. In one example, the nucleic acid array includes at least 2, 5, 10, 20, 30, 40, 50, 100, or more probes for different respective tiling sequences derived from “GLAXO_OA,” “GI_MILD” or “GI_SEVERE”, but not “GLAXO_Normal.” See Attachment F. In another example, the nucleic acid array includes at least 2, 5, 10, 20, 30, 40, 50, 100, or more probes for different respective tiling sequences derived from “GLAXO_Normal,” but not “GLAXO_OA,” “GI_MILD” or “GI_SEVERE.” See Attachment F.

In still another embodiment, a nucleic acid array of the present invention includes at least 5,028 probes, and each probe can hybridize under stringent or nucleic acid array hybridization conditions to a different respective tiling sequence selected from Attachment C, or the complement thereof. In a further embodiment, a nucleic acid array of the present invention comprises at least one probe for each tiling sequence selected from Attachment C.

Multiple probes can be included in the nucleic acid arrays of the present invention for detecting the same tiling sequence. For instance, at least 2, 5, 10, 15, 20, 25, 30 or more different probes can be used for detecting the same tiling sequence selected from Attachment C. In one embodiment, a nucleic acid array of the present invention includes at least 30, 40, 50, or 60 different probes for each tiling sequence of interest. In another embodiment, a nucleic acid array of the present invention includes 25-39 probes for each tiling sequence of interest.

Each probe can be attached to a different respective discrete region on a nucleic acid array. Alternatively, two or more different probes can be attached to the same discrete region. The concentration of one probe with respect to the other probe or probes in the same region may vary according to the objectives and requirements of the particular experiment. In one embodiment, different probes in the same region are present in approximately equimolar ratio.

Preferably, probes for different tiling sequences are attached to different discrete regions on a nucleic acid array. In some applications, probes for different tiling sequences are attached to the same discrete region.

As discussed above, the length of each probe on a nucleic acid array of the present invention can be selected to achieve the desirable hybridization effects. For instance, each probe can include or consist of 15, 20, 25, 30, 35, 40, 45, 50, 60, 70, 80, 90, 100 or more consecutive nucleotides. In one embodiment, each probe consists of 25 consecutive nucleotides. In another embodiment, a nucleic acid array of the present invention includes each and every oligonucleotide probe selected from Attachment E.

The nucleic acid arrays of the present invention can also include control probes which can hybridize under stringent or nucleic acid array hybridization conditions to respective control sequences, or the complements thereof. Suitable control sequences for the present invention are illustrated in Attachment G. Like the parent sequences, each control sequence in Attachment G has a respective SEQ ID NO and a header that includes the qualifier (starting with “wyeHumanOAla”) and other information of the control sequence.

In a preferred embodiment, the nucleic acid arrays of the present invention comprise a perfect mismatch probe for each perfect match probe on the nucleic acid arrays. A perfect mismatch probe has the same sequence as the perfect match probe except for a homomeric substitution (A to T, T to A, G to C, and C to G) at or near the center of the perfect mismatch probe. For instance, if the perfect match probe has 2n nucleotide residues, the homomeric substitution in the perfect mismatch probe is either at the n or n+1 position, but not at both positions. If the perfect match probe has 2n+1 nucleotide residues, the homomeric substitution in the perfect mismatch probe is at the n+1 position. The center location of the mismatched residue is more likely to destabilize the duplex formed with the target sequence under the hybridization conditions. Each perfect match probe and its perfect mismatch probe can be stably attached to different discrete regions on a nucleic acid array of the present invention.

D. Applications

The nucleic acid arrays of the present invention can be used to detect or monitor the expression profiles of human protease or osteoarthritis genes. The nucleic acid arrays of the present invention can also be used to identify or evaluate compounds that can modulate the expression or function of human protease or osteoarthritis genes. In addition, the nucleic acid arrays of the present invention can be used to screen for drug candidates capable of modulating expression of human protease or osteoarthritis genes.

Protocols for conducing nucleic acid array analysis are well known in the art. Exemplary protocols include those provided by Affymetrix in connection with the use of its GeneChip arrays. Samples amenable to nucleic acid array hybridization can be prepared from human cartilage or other tissues. As used herein, “tissue” includes any cell preparations. Thus, a cartilage cell preparation is also considered a cartilage tissue in the present invention.

The sample for hybridization to a nucleic acid array can be either RNA (e.g., mRNA or cRNA) or DNA (e.g., cDNA). Various methods are available for isolating RNA from tissues. These methods include, but are not limited to, RNeasy kits (provided by QIAGEN), MasterPure kits (provided by Epicentre Technologies), and TRIZOL (provided by Gibco BRL). The RNA isolation protocols provided by Affymetrix can also be used.

The isolated RNA preferably is amplified and/or labeled before being hybridized to a nucleic acid array. Suitable RNA amplification methods include, but are not limited to, reverse transcriptase PCR, isothermal amplification, ligase chain reaction, and Qbeta replicase method. The amplification products can be either cDNA or cRNA. In one embodiment, the isolated mRNA is reverse transcribed to cDNA using a reverse transcriptase and a primer consisting of oligo d(T) and a sequence encoding the phage T7 promoter. The cDNA is single stranded. The second strand of the cDNA can be synthesized using a DNA polymerase, combined with an RNase to break up the DNA/RNA hybrid. After synthesis of the double stranded cDNA, T7 RNA polymerase is added to transcribe cRNA from the second strand of the doubled stranded cDNA. In one embodiment, the originally isolated RNA is hybridized to a nucleic acid array without amplification.

cDNA, cRNA, or other nucleic acid samples can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. The labeling moieties include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Nucleic acid samples can be fragmented before being labeled with detectable moieties. Exemplary methods for fragmentation include, for example, heat and/or ion-mediated hydrolysis.

Hybridization reactions can be performed in absolute or differential hybridization formats. In the absolute hybridization format, polynucleotides derived from one sample are hybridized to the probes in a nucleic acid array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from nucleic acid arrays can be analyzed using commercially available software, such as those provided by Affymetrix or Agilent Technologies. Controls, such as for scan sensitivity, probe labeling and cDNA or cRNA quantitation, are preferably included in the hybridization experiments. Hybridization signals can be scaled or normalized before being subject to further analysis. For instance, hybridization signals for each individual probe can be normalized to take into account variations in hybridization intensities when more than one array is used under similar test conditions. Hybridization signals can also be normalized using the intensities derived from internal normalization controls contained on each array. In addition, genes with relatively consistent expression levels across the samples can be used to normalize the expression levels of other genes. In one embodiment, probes for certain maintenance genes are included in a nucleic acid array of the present invention. These genes are chosen because they show stable levels of expression across a diverse set of tissues. Hybridization signals can be normalized and/or scaled based on the expression levels of these maintenance genes.

In a preferred embodiment, probes for certain exogenous transcripts are included in a nucleic acid array of the present invention. These transcripts can be chosen such that they show no similarity to eukaryotic transcripts. In one specific example, eleven exogenous transcripts at different known concentrations are spiked in to each sample. The array is first scaled to a trimmed-mean target value of 100. Based on the scaled hybridization signal of these eleven probe sets, a standard curve can be drawn such that all transcripts present in the sample can be converted from a signal value to a more meaningful concentration value. In another specific example, a standard curve correlating the signal value read off of the array and known frequency (molarity) can be generated when the array image is read and the probe set expression values are generated. From this standard curve, each signal value can then be converted to a parts per million or picomolarity value. The exogenous controls spiked into each sample can include, for instance, E. coli BioB-5, E. coli BioB-M, E. coli BioB-3, E. coli BioC-5, E. coli BioC-3, E. coli BioD-3, Bacteriophage P1 Cre-5, Bacteriophage P1 Cre-3, E. coli Dap-5, B. subtilis Dap-M, and B. subtilis Dap-3. These transcripts can be monitored by control probe sets as discussed below.

The nucleic acid arrays of the present invention can be used to identify compounds that are capable of modulating the expression of human protease or osteoarthritis genes. High-throughput screen methods can be employed. Typically, a compound of interest is first contacted with a cell preparation, such as a cartilage cell preparation. mRNA is extracted from the cell preparation and then hybridized to a nucleic acid array of the present invention. Hybridization signals are compared before and after the treatment with the compound to determine if the compound can modulate the expression of any human protease and/or osteoarthritis genes.

The compound thus identified can be any type of gene modulators. In one embodiment, the compound can bind to the promoter sequence of a human protease or osteoarthritis gene, thereby suppressing or enhancing the transcription of the gene. In another embodiment, the compound modulates the activity of a transcription factor, which in turn controls the expression of the human protease or osteoarthritis gene(s). In yet another embodiment, the compound regulates the degradation, splicing or other modifications of the RNA transcript of the human protease or osteoarthritis gene(s). In a further embodiment, the compound affects the expression or function of another protein which is involved in a cascade regulation of the human protease or osteoarthritis gene(s).

Any in vitro or in vivo assay system can be employed to identify modulators of human protease or osteoarthritis genes. Exemplary assay systems include, but are not limited to, in vitro transcription and translation systems, cell lines, primary cell cultures, and tissue cultures.

Any type of compounds can be evaluated using the present invention. For instance, the compound can be a small molecule, an antibody, a toxin, or a naturally-occurring factor or an analog thereof. Exemplary naturally-occurring factors include, but are not limited to, endocrine factors, paracrine factors, autocrine factors, intracellular factors, and factors interacting with cell receptors. In one embodiment, the compound of interest is an antisense RNA or a double stranded RNA having RNA interference effect (RNAi). Once a lead compound is identified, its derivatives or analogs can be further screened or tested for the optimal modulation effect.

The effect of a compound of interest on the expression of human protease or osteoarthritis genes can also be evaluated in humans or animal models. For instance, the compound of interest can be administered to a human or an animal model. A nucleic acid sample is prepared from the human or animal model, and then hybridized to a nucleic acid array of the present invention. Hybridization signals are analyzed to determine the effect of the compound on the expression of human protease or osteoarthritis genes. Preferably, the animal models are selected such that the false negative and false positive rates are relatively low. Exemplary animal models include primates.

The compound can be administered to the human or animal model via any route of administration. Exemplary routes of administration include parenteral, intravenous, intradermal, subcutaneous, oral, inhalation, transdermal, transmucosal, and rectal administration. The compound can be formulated in a pharmaceutical solution or suspension compatible with the intended route of administration. For instance, solutions or suspensions suitable for parenteral, intradermal, or subcutaneous application can include the following components: a sterile diluent such as water and saline solution, a synthetic solvent such as propylene glycol, antibacterial agents such as benzyl alcohol or methyl parabens, antioxidants such as ascorbic acid or sodium bisulfate, chelating agents such as ethylenediaminetetraacetic acid, buffers such as acetates, citrates or phosphates, and/or agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

In addition, the nucleic acid arrays of the present invention can be used to evaluate the effect of a compound on the function of human protease or osteoarthritis genes. For instance, a human protease or osteoarthritis gene may be involved in the regulation of the expression of another human protease or osteoarthritis gene. By monitoring the expression level of the latter gene, the modulation effect of a compound on the function of the former gene can be determined.

Furthermore, the nucleic acid arrays of the present invention can be used to evaluate the effect of a drug candidate on treating osteoarthritis. The drug candidate can be administered to a human affected with osteoarthritis via any suitable route. A tissue of interest, such as a cartilage tissue, is then isolated from the human. A nucleic acid sample is prepared from the tissue and hybridized to a nucleic acid array of the present invention. Hybridization signals are analyzed to determine the effect of the drug candidate on the gene expression profiles in the tissue of interest. Preferably, the drug candidate can return the expression levels of osteoarthritis genes to their normal levels.

The present invention also features protein arrays for expression profiling of human protease and/or osteoarthritis genes. Each protein array of the present invention includes probes which can specifically bind to protein products of respective human protease or osteoarthritis genes. Examples of human protease or osteoarthritis genes include those that encode the tiling sequences of Attachment C.

In one embodiment, the probes on a protein array of the present invention are antibodies. Many of these antibodies can bind to the corresponding target proteins with an affinity constant of at least 10⁴ M⁻¹, 10⁵ M⁻¹, 10⁶ M⁻¹, 10⁷ M⁻¹, or stronger. Suitable antibodies for the present invention include, but are not limited to, polyclonal antibodies, monoclonal antibodies, chimeric antibodies, single chain antibodies, synthetic antibodies, Fab fragments, or fragments produced by a Fab expression library. Other peptides, scaffolds, antibody mimics, high-affinity binders, or protein-binding ligands can also be used to construct the protein arrays of the present invention.

Numerous methods are available for immobilizing antibodies or other probes on a protein array of the present invention. Examples of these methods include, but are not limited to, diffusion (e.g., agarose or polyacrylamide gel), surface absorption (e.g., nitrocellulose or PVDF), covalent binding (e.g., silanes or aldehyde), or non-covalent affinity binding (e.g., biotin-streptavidin). Examples of protein array fabrication methods include, but are not limited to, ink-jetting, robotic contact printing, photolithography, or piezoelectric spotting. The method described in MacBeath and Schreiber, SCIENCE, 289: 1760-1763 (2000), which is incorporated herein by reference, can also be used. Suitable substrate supports for a protein array of the present invention include, but are not limited to, glass, membranes, mass spectrometer plates, microtiter wells, silica, or beads.

The protein-coding sequence of a human protease or osteoarthritis gene can be determined by a variety of methods. For instance, the protein-coding sequences can be extracted from the corresponding tiling or parent sequences by using an open reading frame (ORF) prediction program. Examples of ORF prediction programs include, but are not limited to, GeneMark (provided by the European Bioinformatics Institute), Glimmer (provided by TIGR), and ORF Finder (provided by NCBI). Many protein sequences can also be obtained from Entrez or other sequence databases by BLAST searching the corresponding tiling or parent sequences against these databases. The protein-coding sequences thus obtained can be used to prepare antibodies or other protein-binding agents.

In addition, the present invention contemplates collections of polynucleotides. In one embodiment, a polynucleotide collection of the present invention comprises at least one set of probes capable of hybridizing under stringent or nucleic acid array hybridization conditions to a tiling sequence selected from Attachment C, or the complement thereof. In another embodiment, a polynucleotide collection of the present invention comprises at least 2, 5, 10, 50, 100, 500, 1,000 or more sets of probes, and each probe set is capable of hybridizing under stringent or nucleic acid array hybridization conditions to a different respective tiling sequence selected from Attachment C, or the complement thereof. In yet another embodiment, a polynucleotide collection of the present invention includes at least 1, 2, 5, 10, 50, 100, 500, 1,000, 5,000, or more tiling sequences selected from Attachment C, or the complements thereof. In still yet another embodiment, a polynucleotide collection of the present invention contains at least 1, 2, 5, 10, 50, 100, 500, 1,000, 5,000, or more sequence selected from SEQ ID NOs: 1-5,235, or the complements thereof.

It should be understood that the above-described embodiments and the following examples are given by way of illustration, not limitation. Various changes and modifications within the scope of the present invention will become apparent to those skilled in the art from the present description.

### E. EXAMPLES

### Example 1

### Nucleic Acid Array

The tiling sequences depicted in Attachment C were submitted to Affymetrix for custom array design. Affymetrix selected probes for each tiling sequence using its probe-picking algorithm. Non-ambiguous probes with 25 bases in length were selected. Thirty-nine probe-pairs were requested for each tiling sequence with a minimum number of acceptable probe-pairs set to twenty-five. The final array was directed to 5,028 human transcripts and contained 198,286 perfect match probes and 198,286 mismatch probes, including 102 exogenous and endogenous control probe sets. These probes are shown in Attachment H.

The probes in Attachment H are perfect match probes and correspond to SEQ ID NOs: 5,338-203,623, respectively. Each probe in Attachment H has a qualifier (“Header”) which is identical to the qualifier of the corresponding tiling sequence from which the probe is derived. The strandedness of each probe (“Target Strandedness”) is also demonstrated.

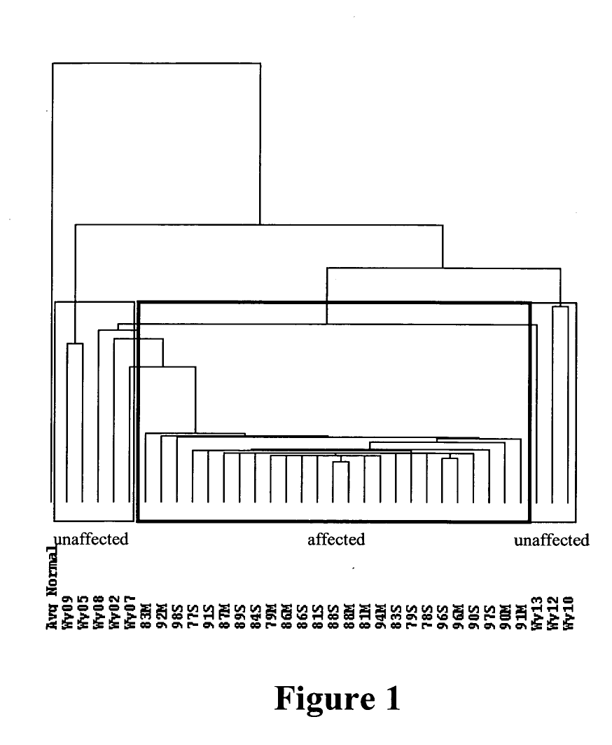

FIGURE 1 shows an Eisen cluster of transcriptional profiling data generated using the above-described custom array. Data was scale frequency normalized. Only those qualifiers with at least 1 present call in any sample were used for the cluster analysis. Data were log transformed, and hierarchical clustering was done using the average linkage clustering function on the arrays. Levels of all expressed genes strongly segregate samples affected by osteoarthritis (OA) from unaffected cartilage samples.

### Example 2

### Nucleic Acid Array Hybridization

10 μg of biotin-labeled sample DNA/RNA is diluted in 1×MES buffer with 100 μg/ml herring sperm DNA and 50 μg/ml acetylated BSA. To normalize arrays to each other and to estimate the sensitivity of the nucleic acid arrays, in vitro synthesized transcripts of control genes are included in each hybridization reaction. The abundance of these transcripts can range from 1:300,000 (3 ppm) to 1:1000 (1000 ppm) stated in terms of the number of control transcripts per total transcripts. As determined by the signal response from these control transcripts, the sensitivity of detection of the arrays can range, for example, between about 1:300,000 and 1:100,000 copies/million. Labeled DNA/RNA are denatured at 99° C. for 5 minutes and then 45° C. for 5 minutes and hybridized to the nucleic array of Example 1. The array is hybridized for 16 hours at 45° C. The hybridization buffer includes 100 mM MES, 1 M [Na⁺], 20 mM EDTA, and 0.01% Tween 20. After hybridization, the cartridge(s) is washed extensively with wash buffer (6×SSPET), for instance, three 10-minute washes at room temperature. The washed cartridge(s) is then stained with phycoerythrin coupled to streptavidin.

12×MES stock contains 1.22 M MES and 0.89 M [Na⁺]. For 1000 ml, the stock can be prepared by mixing 70.4 g MES free acid monohydrate, 193.3 g MES sodium salt and 800 ml of molecular biology grade water, and adjusting volume to 1000 ml. The pH should be between 6.5 and 6.7. 2× hybridization buffer can be prepared by mixing 8.3 ml of 12×MES stock, 17.7 ml of 5 M NaCl, 4.0 ml of 0.5 M EDTA, 0.1 ml of 10% Tween 20 and 19.9 ml of water. 6×SSPET contains 0.9 M NaCl, 60 mM NaH₂PO₄, 6 mM EDTA, pH 7.4, 05% Triton X-100. In some cases, the wash buffer can be replaced with a more wash buffer. 1000 ml stringent wash buffer can be prepared by mixing 83.3 ml of stock, 5.2 ml of 5 M NaCl, 1.0 ml of 10% Tween 20 and 910.5 ml of water.

The foregoing description of the present invention provides illustration and ion, but is not intended to be exhaustive or to limit the invention to the precise one d. Modifications and variations are possible consistent with the above teachings or acquired from practice of the invention. Thus, it is noted that the scope of the n is defined by the claims and their equivalents.

1. A nucleic acid array comprising one or more substrate supports which are stably associated with polynucleotide probes, wherein a substantial portion of all polynucleotide probes that are stably associated with said one or more substrate supports is capable of hybridizing under stringent or nucleic acid array hybridization conditions to human genes, and each said human gene is selected from the group consisting of protease genes and genes that are differentially expressed in osteoarthritic human cartilage cells as compared to osteoarthritis-free human cartilage cells.

2. The nucleic acid array according to claim 1, wherein the substantial portion of all polynucleotide probes comprises one or more first probe sets, and each said first probe set is capable of hybridizing under stringent or nucleic acid array hybridization conditions to a gene whose average expression level in osteoarthritic human cartilage cells is higher than that in osteoarthritis-free human cartilage cells.

3. The nucleic acid array according to claim 2, wherein the substantial portion of all polynucleotide probes further comprises one or more second probe sets, and each said second probe set is capable of hybridizing under stringent or nucleic acid array hybridization conditions to a gene whose average expression level in osteoarthritis-free human cartilage cells is higher than that in osteoarthritic human cartilage cells.

4. The nucleic acid array according to claim 3, wherein the substantial portion of all polynucleotide probes further comprises one or more third probe sets, and each said third probe set is capable of hybridizing under stringent or nucleic acid array hybridization conditions to a human protease gene.

5. The nucleic acid array of claim 4, wherein the substantial portion of all polynucleotide probes comprises at least 10 first probe sets, each of which is capable of hybridizing under stringent or nucleic acid array hybridization conditions to a different respective gene whose expression level is substantially higher in osteoarthritic human cartilage cells than in osteoarthritis-free human cartilage cells, wherein the substantial portion of all polynucleotide probes further comprises at least 10 second probe sets, each of which is capable of hybridizing under stringent or nucleic acid array hybridization conditions to a different respective gene whose expression level is substantially higher in osteoarthritis-free human cartilage cells than in osteoarthritic human cartilage cells, and wherein the substantial portion of all polynucleotide probes further comprises at least 10 third probe sets, each of which is capable of hybridizing under stringent or nucleic acid array hybridization conditions to a different respective human protease gene.

6. The nucleic acid array of claim 4, wherein the substantial portion of all polynucleotide probes comprises at least 100 first probe sets, each of which is capable of hybridizing under stringent or nucleic acid array hybridization conditions to a different respective gene whose expression level is substantially higher in osteoarthritic human cartilage cells than in osteoarthritis-free human cartilage cells, wherein the substantial portion of all polynucleotide probes further comprises at least 100 second probe sets, each of which is capable of hybridizing under stringent or nucleic acid array hybridization conditions to a different respective gene whose expression level is substantially higher in osteoarthritis-free human cartilage cells than in osteoarthritic human cartilage cells, and wherein the substantial portion of all polynucleotide probes further comprises at least 100 third probe sets, each of which is capable of hybridizing under stringent or nucleic acid array hybridization conditions to a different respective human protease gene.

7. The nucleic acid array of claim 4, wherein the substantial portion of all polynucleotide probes includes at least 25% of all polynucleotide probes that are stably associated with said one or more substrate supports.

8. The nucleic acid array of claim 4, wherein the substantial portion of all polynucleotide probes includes at least 45% of all polynucleotide probes that are stably associated with said one or more substrate supports.

9. The nucleic acid array according to claim 1, wherein the substantial portion of all polynucleotide probes comprises at least one probe set, and each said probe set is capable of hybridizing under stringent or nucleic acid array hybridization conditions to a tiling sequence selected from Attachment C, or the complement thereof.

10. The nucleic acid array according to claim 1, wherein the substantial portion of all polynucleotide probes comprises at least 10 probe sets, each of which is capable of hybridizing under stringent or nucleic acid array hybridization conditions to a different respective tiling sequence selected from Attachment C, or the complement thereof.

11. The nucleic acid array according to claim 1, wherein the substantial portion of all polynucleotide probes comprises at least 1,000 probe sets, each of which is capable of hybridizing under stringent or nucleic acid array hybridization conditions to a different respective tiling sequence selected from Attachment C, or the complement thereof.

12. The nucleic acid array according to claim 1, wherein the substantial portion of all polynucleotide probes comprises each and every polynucleotide probe selected from Attachment E.

13. The nucleic acid array according to claim 12, comprising a perfect mismatch probe for each polynucleotide probe selected from Attachment E.

14. A method of screening for candidate drugs capable of modulating expression of human protease or osteoarthritis genes comprising the steps of:
(a) preparing a first nucleic acid sample from a human affected by osteoarthritis;
(b) hybridizing the first nucleic acid sample to a first nucleic acid array as in any one of claims 1-4;
(c) detecting a first set of hybridization signals;
(d) treating the human with a candidate drug;
(e) repeating steps (a)-(c) with a second nucleic acid sample from the treated human and a second nucleic acid array identical to the first array to obtain a second set of hybridization signals; and
(f) comparing the first and second sets of hybridization signals,
wherein any change in expression level of at least one protease gene, and/or one gene differentially expressed in osteoarthritic human cartilage cells as compared to osteoarthritis-free human cartilage cells, identifies the candidate drug as one that modulates expression of human protease or osteoarthritis genes.

15. The method according to claim 14, wherein the first and second nucleic acid samples are prepared from cartilage tissues of the human.

16. A method of screening for candidate drugs capable of modulating expression of human protease or osteoarthritis genes comprising the steps of:
(a) preparing a first nucleic acid sample from a cell or tissue affected by osteoarthritis;
(b) hybridizing the first nucleic acid sample to a first nucleic acid array as in any one of claims 1-4;
(c) detecting a first set of hybridization signals;
(d) treating the cell or tissue with a candidate drug;
(e) repeating steps (a)-(c) with a second nucleic acid sample from the treated cell or tissue and a second nucleic acid array identical to the first array to obtain a second set of hybridization signals; and
(f) comparing the first and second sets of hybridization signals,
wherein any change in expression level of at least one protease gene, and/or one gene differentially expressed in osteoarthritic human cartilage cells as compared to osteoarthritis-free human cartilage cells, identifies the candidate drug as one that modulates expression of human protease or osteoarthritis genes.

17. The method according to claim 16, wherein the cell or tissue is prepared from a human cartilage tissue.

18. A nucleic acid array comprising a plurality of probe sets, wherein each said probe set is capable of hybridizing under stringent or nucleic acid array hybridization conditions to a different respective tiling sequence selected from Attachment C, or the complement thereof.

19. The nucleic acid array according to claim 18, wherein said plurality of probe sets comprises at least 100 probe sets.

20. The nucleic acid array according to claim 18, wherein said plurality of probe sets comprises at least 5,028 probe sets.

21. The nucleic acid array according to claim 18, wherein for each tiling sequence selected from Attachment C, said plurality of probe sets comprises at least one probe set capable of hybridizing under stringent or nucleic acid array hybridization conditions to that tiling sequence, or the complement thereof.

22. The nucleic acid array according to claim 18, wherein said plurality of probe sets comprises a substantial portion of all polynucleotide probes that are stably associated with the nucleic acid array.

23. A polynucleotide collection comprising a probe set capable of hybridizing under stringent or nucleic acid array hybridization conditions to a tiling sequence selected from Attachment C, or the complement thereof.

24. A polynucleotide collection comprising at least one tiling sequence selected from Attachment C, or the complement thereof.

25. A polynucleotide collection comprising at least one sequence selected from SEQ ID NOs: 1-5,235, or the complement thereof.

26. A probe array comprising one or more substrate supports, wherein a substantial portion of all probes that are stably associated with said one or more substrate supports is capable of specifically binding to protein products of human protease or osteoarthritis genes.
